(19) United States
(12) Patent Application Publication
(10) Pub. No.: US 2020/0113643 A1
(43) Pub. Date: Apr. 16, 2020

(54) SHEATHING AND LUBRICATING DEVICE AND METHOD FOR LUBRICATING AND DISINFECTING A SURGICAL INSTRUMENT

(71) Applicant: Robert Backstein, North York (CA)

(72) Inventor: Robert Backstein, North York (CA)

(21) Appl. No.: 16/626,487

(22) PCT Filed: Jul. 5, 2018
(86) PCT No.: PCT/IB2018/054984

§ 371 Date: Dec. 24, 2019

Related U.S. Application Data
(60) Provisional application No. 62/529,544, filed on Jul. 7, 2017.

(51) Int. Cl. A61B 46/10; A61B 90/70; A61L 2/18

(52) U.S. Cl. CPC A61B 46/10; A61B 90/70; A61L 2202/24; A61L 2202/15; A61L 2/18

(57) ABSTRACT

A protective sheathing and lubricating device for use with a surgical instrument, including an elongate element arranged along a longitudinal axis and having a distal end and a proximal end; the elongate element is configured to be axially compressible upon exertion of force onto the proximal end.

### REFERENCE TO RELATED APPLICATIONS

Reference is hereby made to U.S. Provisional Patent Application 62/529,544, filed Jul. 7, 2017 and entitled “PROTECTIVE SHEATHING DEVICE AND METHOD OF DISINFECTING FOR A SURGICAL INSTRUMENT”, the disclosure of which is incorporated by reference in its entirety and priority of which is hereby claimed pursuant to 37 CFR 1.78(a) (4) and (5)(i).

### FIELD OF THE INVENTION

The present invention relates to surgical instruments and more particularly to surgical instruments providing for lubrication and for sterile environment.

### BACKGROUND OF THE INVENTION

Various surgical instruments, such as surgical cannulas and endoscopes, are typically inserted into the body of a patient via a small incision in order to perform a surgical procedure, such as a liposuction, for example. It is known that the surgical instruments are prone to contamination during the surgical procedure, due to their long dimensions and potential contact with non-sterile parts of the surgical field. Insertion of a contaminated surgical instrument into the body tissues of the patient may lead to septic effects for the patient.

It is also known that displacement of a surgical instrument through the small incision during surgical procedure can lead to friction burns on the skin of the patient.

There is thus a need for a protective sheathing and lubricating device for the various surgical instruments.

### SUMMARY OF THE INVENTION

The present invention seeks to provide a protective sheathing and lubricating device for surgical instruments.

There is thus provided in accordance with an embodiment of the present invention a protective sheathing and lubricating device for use with a surgical instrument, including a collapsible sheath arranged along a longitudinal axis and having a distal end, a proximal end and an interior volume; the collapsible sheath is configured to be axially compressible upon exertion of force onto the proximal end; the collapsible sheath is further configured to automatically assume an at-rest operative state upon release of the force.

Preferably, the protective sheathing and lubricating device also includes a biasing element, which extends within the interior volume of the collapsible sheath along the longitudinal axis. Further preferably, the protective sheathing and lubricating device also includes a tip element, which is fixedly attached to at least one of the distal end and the proximal end of the collapsible sheath.

Still further preferably, the biasing element is fixedly attached to the tip element. Yet further preferably, the collapsible sheath is made of a resilient material.

In accordance with an embodiment of the present invention, the biasing element is a compression spring. Alternatively, the biasing element is a mesh sleeve. Preferably, The tip element is a sponge foam.

In accordance with an embodiment of the present invention, a protective sheathing and lubricating device for use with a surgical instrument, including a biasing element arranged along a longitudinal axis and having a distal end, a proximal end; the biasing element is configured to be axially compressible upon exertion of force onto the proximal end; the biasing element is further configured to automatically assume an at-rest operative state upon release of the force.

Preferably, the protective sheathing and lubricating device also includes a collapsible sheath mounted over the biasing element and arranged coaxially therewith. Further preferably, the protective sheathing and lubricating device also includes a tip element, which is fixedly attached to at least one of the distal end and the proximal end of at least one of the collapsible sheath and the biasing element.

Preferably, the biasing element is a compression spring. Alternatively, the biasing element is a mesh sleeve. Further alternatively, the biasing element is a sponge foam. Additionally, the tip element is a sponge foam.

In accordance with an embodiment of the present invention, a protective sheathing and lubricating device for use with a surgical instrument, including a biasing element arranged along a longitudinal axis and having a distal end and a proximal end; a tip element coupled with the biasing element; the biasing element is configured to be axially compressible upon exertion of force onto the proximal end.

Preferably, the tip element has an annular portion and an elongated portion extending generally transversely with respect to the annular portion. Further preferably, the elongated portion is conical.

In accordance with an embodiment of the present invention, a method of covering and disinfecting a surgical instrument during insertion thereof into a surgical site, including the steps of: providing a protective sheathing and lubricating device, having a tubular collapsible sheath having a distal end, a proximal end, an interior volume and extending along a longitudinal axis; inserting an elongated shaft of the surgical instrument into the interior volume of the collapsible sheath, such that the entire longitudinal extent of the elongated shaft is covered by the collapsible sheath; displacing the surgical instrument distally relative to the collapsible sheath, thereby axially compressing the collapsible sheath; and thereafter displacing the surgical instrument proximally relative to the collapsible sheath, thereby automatically causing the collapsible sheath to assume an at-rest operative state, in which the collapsible sheath covers the entire longitudinal extent of the elongated shaft.

Preferably, the protective sheathing and lubricating device also includes a biasing element, which extends within the interior volume of the collapsible sheath along the longitudinal axis. Further preferably, the protective sheathing and lubricating device also includes a tip element, which is fixedly attached to at least one of the distal end and the proximal end of the collapsible sheath.

Still further preferably, the biasing element is fixedly attached to the tip element. Yet further preferably, the collapsible sheath is made of a resilient material.

Preferably, the biasing element is a compression spring. Further preferably, the tip element is a sponge foam. Additionally, the tip element is configured to be saturated with a disinfecting agent; and at least a portion of the surgical instrument is disinfected during the displacement of the surgical instrument. Alternatively, or additionally, the biaising element is configured to be saturated with a disinfecting agent; and at least a portion of the surgical instrument is disinfected during the displacement of the surgical instrument.

Preferably, the disinfecting agent is iodine. Further preferably, the disinfection occurs by means of contact between the at least portion of surgical instrument and the tip element. Still further preferably, the disinfection occurs by means of contact between the at least portion of surgical instrument and the biasing element. Yet further preferably, the disinfection occurs by means of exudation of the disinfecting agent from the tip element to the at least portion of the surgical instrument. Still further preferably, the disinfection occurs by means of exudation of the disinfecting agent from the biasing element to the at least portion of the surgical instrument.

In accordance with an embodiment of the present invention, a method of covering and disinfecting a surgical instrument during insertion thereof into a surgical site, including the steps of: providing a protective sheathing and lubricating device, having a biasing element arranged along a longitudinal axis and having a distal end and a proximal end; inserting an elongated shaft of the surgical instrument through the biasing element, such that the entire longitudinal extent of the elongated shaft is covered by the biasing element; displacing the surgical instrument distally relative to the biasing element, thereby axially compressing the biasing element; and thereafter displacing the surgical instrument proximally relative to the biasing element, thereby automatically causing the biasing element to assume an at-rest operative state, in which the biasing element covers the entire longitudinal extent of the elongated shaft.

Preferably, the protective sheathing and lubricating device also including a sheathing element mounted over the biasing element and being coaxial therewith. Further preferably, the protective sheathing and lubricating device also including a tip element, which is fixedly attached to at least one of the distal end and the proximal end of at least one of the collapsible sheath and the biasing element. Still further preferably, the tip element is configured to be saturated with a disinfecting agent; and at least a portion of the surgical instrument is disinfected during the displacement of the surgical instrument. Yet further preferably, the biasing element is configured to be saturated with a disinfecting agent; and at least a portion of the surgical instrument is disinfected during the displacement of the surgical instrument.

Preferably, the tip element includes an annular portion and an elongated portion extending generally transversely with respect to the annular portion. Further preferably, the elongated portion is conical. Still further preferably, the tip element is configured to be pre-cut to a desired length to form an opening within the elongate portion for insertion of at least a portion of the surgical instrument therethrough.

In accordance with an embodiment of the present invention, a method of disinfecting or lubricating a surgical instrument during insertion thereof into a surgical site, including the steps of: providing a protective sheathing and lubricating device, having an elongate element arranged along a longitudinal axis and having a distal end and a proximal end; saturating at least a portion of the sheathing and lubricating device within a disinfecting agent; inserting at least a portion of the surgical instrument into the elongate element, such that at least a portion of the surgical instrument is disinfected by the protective sheathing and lubricating device.

Preferably, the protective sheathing and lubricating device also includes a tip element, which is coupled to the elongate element. Further preferably, the disinfection occurs by means of contact between the at least portion of surgical instrument and at least a portion of the tip element. Still further preferably, the disinfection occurs by means of exudation of the disinfecting agent from at least a portion of the tip element to the at least portion of the surgical instrument. Yet further preferably, the tip element is adapted to be at least partially inserted into the surgical site.

In accordance with an embodiment of the present invention, a protective sheathing and lubricating device for use with a surgical instrument, including an elongate element arranged along a longitudinal axis and having a distal end and a proximal end; the elongate element is configured to be axially compressible upon exertion of force onto the proximal end.

Preferably, the protective sheathing and lubricating device also includes a tip element coupled with the elongate element. Still preferably, the protective sheathing and lubricating device also includes a collapsible sheath mounted over the elongate element and arranged coaxially therewith.

Preferably, the elongate element is a compression spring. Alternatively, the elongate element is a mesh sleeve. Further alternatively, the elongate element is a sponge foam.

Preferably, the tip element is a sponge foam. Further preferably, the tip element serves as a port, configured to be at least partially inserted into a patient's body. Still further preferably, the tip element is configured to be saturated in a fluid; and at least a portion of the surgical instrument is lubricated during displacement thereof through the tip element. Alternatively, or additionally, the elongate element is configured to be saturated in a fluid; and at least a portion of the surgical instrument is lubricated during displacement thereof through the elongate element.

Preferably, the lubrication occurs by means of contact between the at least portion of surgical instrument and the tip element. Additionally, the lubrication occurs by means of contact between the at least portion of surgical instrument and the elongate element. Alternatively, or additionally, the lubrication occurs by means of exudation of the fluid from the tip element to the at least portion of the surgical instrument. Further alternatively, or additionally, the lubrication occurs by means of exudation of the fluid from the elongate element to the at least portion of the surgical instrument.

In accordance with an embodiment of the present invention, a kit, includes at least one surgical instrument having a longitudinal shaft; an elongate element arranged along a longitudinal axis and having a distal end and a proximal end, and adapted to be mounted over the longitudinal shaft; and wherein the elongate element is configured to be axially compressible upon exertion of force onto the proximal end.

Preferably, the kit also includes a tip element coupled with the elongate element. Further preferably, the kit also includes a collapsible sheath mounted over the elongate element and arranged coaxially therewith.

### BRIEF DESCRIPTION OF THE DRAWINGS

The present invention will be understood and appreciated more fully from the following detailed description, taken in conjunction with the drawings in which:

FIG. 1 is a simplified pictorial illustration of a protective sheathing and lubricating device constructed and operative in accordance with an embodiment of the present invention;

FIG. 2 is a simplified exploded view illustration of the protective sheathing and lubricating device of FIG. 1;

FIG. 3 is a simplified pictorial illustration of a tip element forming part of the protective sheathing and lubricating device of FIG. 1, according to one embodiment of the present invention;

FIG. 4 is a simplified pictorial illustration of a tip element forming part of the protective sheathing and lubricating device of FIG. 1, according to another embodiment of the present invention;

FIGS. 5A-5C are simplified pictorial illustrations of a tip element forming part of the protective sheathing and lubricating device of FIG. 1, according to still another embodiment of the present invention, the tip element is shown in a closed operative orientation, open operative orientation and with a surgical instrument inserted through the tip element respectively;

FIGS. 6A-6B are a simplified pictorial and sectional illustrations respectively of a tip element forming part of the protective sheathing and lubricating device of FIG. 1, according to yet another embodiment of the present invention, section being taken along lines B-B in FIG. 6A;

FIG. 7 is a simplified sectional illustration of the protective sheathing and lubricating device of FIG. 1, section being taken along lines A-A in FIG. 1;

FIG. 8 is a simplified pictorial illustration showing the protective sheathing and lubricating device of FIG. 1, along with a surgical instrument inserted thereinto, in a preparatory operative orientation as used by a surgeon;

FIG. 9 is a simplified sectional illustration showing the protective sheathing and lubricating device of FIG. 1, along with a surgical instrument inserted thereinto, in a pre-insertion operative orientation as used by a surgeon, section being taken along lines A-A in FIG. 1;

FIG. 10 is a simplified sectional illustration showing the protective sheathing and lubricating device of FIG. 1, along with a surgical instrument inserted thereinto, in a partially-inserted operative orientation as used by a surgeon;

FIG. 11 is a simplified pictorial illustration showing the protective sheathing and lubricating device of FIG. 1, along with a surgical instrument inserted thereinto, in a fully-inserted operative orientation as used by a surgeon;

FIG. 12 is a simplified pictorial illustration showing the protective sheathing and lubricating device of FIG. 1, along with a surgical instrument inserted thereinto, in a retracted operative orientation as used by a surgeon;

FIG. 13 is a simplified sectional illustration of a protective sheathing and lubricating device constructed and operative in accordance with another embodiment of the present invention;

FIG. 14 is a simplified pictorial illustration showing the protective sheathing and lubricating device of FIG. 13, along with a surgical instrument inserted thereinto, in a preparatory operative orientation as used by a surgeon;

FIG. 15 is a simplified sectional illustration showing the protective sheathing and lubricating device of FIG. 13, along with a surgical instrument inserted thereinto, in a pre-insertion operative orientation as used by a surgeon;

FIG. 16 is a simplified sectional illustration showing the protective sheathing and lubricating device of FIG. 13, along with a surgical instrument inserted thereinto, in a partially-inserted operative orientation as used by a surgeon;

FIG. 17 is a simplified pictorial illustration showing the protective sheathing and lubricating device of FIG. 13, along with a surgical instrument inserted thereinto, in a fully-inserted operative orientation as used by a surgeon;

FIG. 18 is a simplified pictorial illustration showing the protective sheathing and lubricating device of FIG. 13, along with a surgical instrument inserted thereinto, in a retracted operative orientation as used by a surgeon.

FIG. 19 is a simplified pictorial illustration of a protective sheathing and lubricating device constructed and operative in accordance with still another embodiment of the present invention;

FIG. 20 is a simplified pictorial illustration showing the protective sheathing and lubricating device of FIG. 19, in a preparatory operative orientation as used by a surgeon;

FIG. 21 is a simplified pictorial illustration showing the protective sheathing and lubricating device of FIG. 19, along with a surgical instrument inserted thereinto, in a partially-inserted operative orientation as used by a surgeon;

FIGS. 22A-22C are respective two simplified pictorial illustrations and a sectional illustration of a tip element forming part of the protective sheathing and lubricating device of FIG. 19, according to another embodiment of the present invention, the tip element in the pictorial illustrations is shown in a preparatory operative orientation, and in a ready for use operative orientation respectively and the tip element is shown in use in the sectional illustration, section being taken along lines C-C in FIG. 22B.

### DETAILED DESCRIPTION OF EMBODIMENTS OF THE INVENTION

The present invention preferably includes a protective sheathing and lubricating device which includes a collapsible sheath, a biasing element and optionally a tip element. During a surgical procedure, a surgical instrument is configured to be inserted into the body of a patient through the protective sheathing and lubricating device, such as to provide isolation of the surgical instrument from external contaminants.

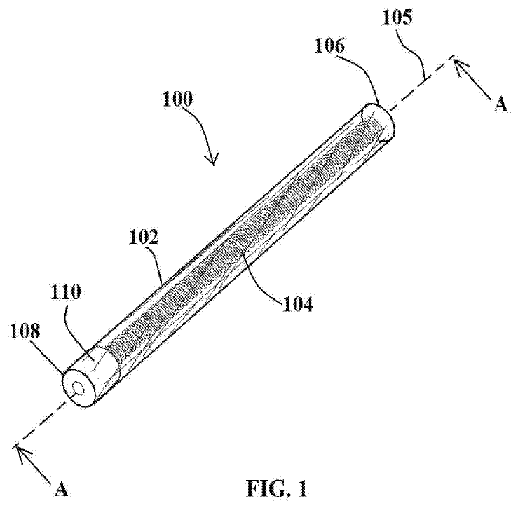

Reference is now made to FIG. 1, which is a simplified pictorial illustration of a protective sheathing and lubricating device constructed and operative in accordance with an embodiment of the present invention.

A protective sheathing and lubricating device 100 is seen in FIG. 1, and includes a collapsible sheath 102 and a biasing element 104, which are arranged coaxially along a mutual longitudinal axis 105. The collapsible sheath 102 defines a proximal end 106 and a distal end 108 and a tip element 110 is preferably positioned adjacent the distal end 108 of the collapsible sheath 102.

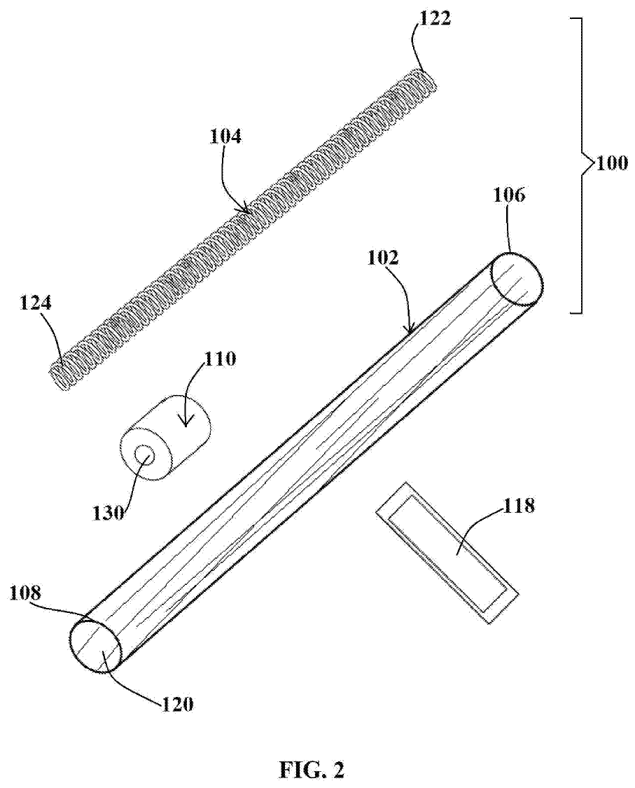

Reference is now made to FIG. 2, which is a simplified exploded view illustration of the protective sheathing and lubricating device 100 of FIG. 1.

It is seen that according to an embodiment of the present invention an adhesive tape 118 is configured to be attached to the proximal end 106 of the collapsible sheath 102 for attachment thereof with a surgical instrument during the surgical procedure.

It is noted that the collapsible sheath 102 is preferably an integrally made tubular element, which is preferably made of a plastic material, and defining an internal volume 120. Alternatively, the collapsible sheath 102 is made of other relatively resilient materials, such as rubber, silicone or fabric.

The biasing element 104 is configured to be inserted into the internal volume 120 of the collapsible sheath 102 and is illustrated as a coil compression spring according to an embodiment of the present invention, however any other type of spring element can be employed in accordance with an embodiment of the present invention, such as for example a collapsible strip of plastic, which is affixed to the outside surface of the collapsible sheath 102. Alternatively, the biasing element 104 is configured as a plastic mesh sleeve, such as for example nylon mesh sleeve. The advantage of using a mesh sleeve biasing element 104 is that it has a relatively small compressed length, in comparison with a compressed length of a coil spring, and therefore minimizes loss of functional length of the medical cannula 350.

The biasing element 104 defines a proximal end 122 and a distal end 124.

The tip element 110 is preferably configured as foam sponge, preferably made of materials such as polyurethane, polyethylene, neoprene or silicone. The tip element 110 is alternatively made of fabric, such as gauze or cotton, plastics, rubbers or other materials.

It is seen in FIG. 2 that the tip element 110 includes a through bore 130 extending along the longitudinal axis 105 according to an embodiment of the present invention.

Reference is now made to FIG. 3, which is a simplified pictorial illustration of the tip element 110 forming part of the protective sheathing and lubricating device 100 of FIG. 1, according to one embodiment of the present invention.

The tip element 110 illustrated in FIG. 3 is preferably integrally made of foam or fabric material and is configured to be mounted adjacent the distal end 108 of the collapsible sheath 102 within the interior volume 120 thereof. Alternatively, the tip element 110 is configured to be attached to the distal end 108 of the collapsible sheath 102, such that the tip element is not enclosed within the internal volume 120 of the collapsible sheath 102, rather extends distally therefrom. The tip element 110 according to one embodiment of the present invention has a through bore 130 extending therethrough and allowing insertion of a portion of surgical instrument therethrough.

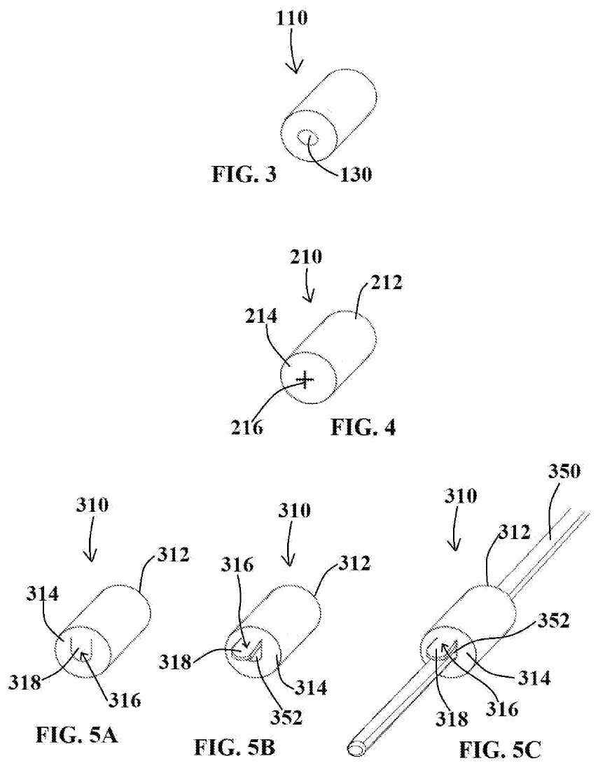

Reference is now made to FIG. 4, which is a simplified pictorial illustration of a tip element 210 forming part of the protective sheathing and lubricating device 100 of FIG. 1, according to another embodiment of the present invention.

The tip element 210 according to another embodiment of the present invention has a proximal end 212 and a distal end 214, which includes a slit-type valve 216. Typically, two intersecting slits are illustrated in FIG. 4, however it is appreciated that any number and shape of slits can be formed at the distal end 214 of tip element 210 in order to seal the tip element 210 when no surgical instrument extends therethrough and provide passage of the surgical instrument into the body of the patient during the surgical procedure.

Reference is now made to FIGS. 5A-5C, which are simplified pictorial illustrations of a tip element 310 forming part of the protective sheathing and lubricating device 100 of FIG. 1, according to still another embodiment of the present invention, the tip element 310 is shown in a closed operative orientation, open operative orientation and with a surgical instrument inserted through the tip element 310 respectively.

The tip element 310 according to still another embodiment of the present invention has a proximal end 312 and a distal end 314, which includes a flap-type valve 316 having a foldable portion 318, which is configured to seal the tip element 310 when no surgical instrument extends therethrough and provide passage of the surgical instrument into the body of the patient during the surgical procedure. The tip element 310 is seen in a closed operative orientation in FIG. 5A, whereas the foldable portion 318 is substantially coplanar with the distal end 314 of the tip element 310.

As seen in FIGS. 5B & 5C, when a surgical instrument, such as a medical cannula 350, is introduced through the tip element 310, the foldable portion 318 is folded preferably distally and allows the medical cannula 350 to extend through a resulting aperture 352 in the distal end 314 of the tip element 310. It is noted that once the medical cannula 350 is removed from the tip element 310, the flap-type valve 316 assumes its normal closed operative orientation, such as shown in FIG. 5A.

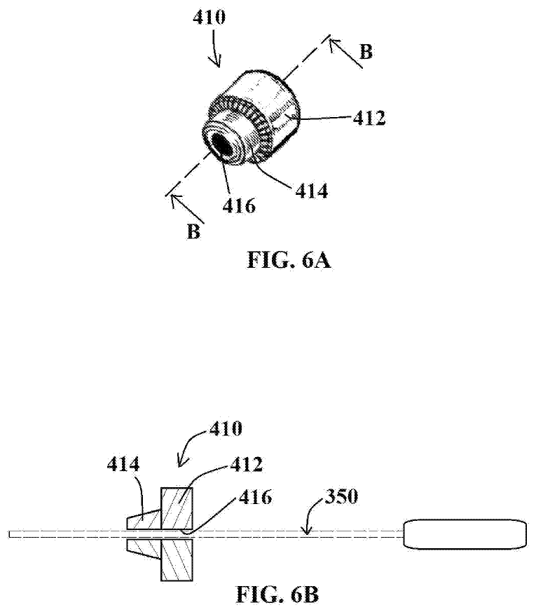

Reference is now made to FIGS. 6A-6B, which are a simplified pictorial and sectional illustrations respectively of a tip element 410 forming part of the protective sheathing and lubricating device 100 of FIG. 1, according to yet another embodiment of the present invention, section being taken along lines B-B in FIG. 6A.

The tip element 410 according to yet another embodiment of the present invention has a proximal portion 412 and a distal portion 414, which are preferably rotatable relative to each other. A through bore 416 is formed through the tip element 410, and the diameter of the bore 416 is preferably adjusted using a known chuck-type mechanism upon rotation of the proximal portion 412 relative to the distal portion 414. It is noted that the tip element 410 may be positioned in a closed operative orientation by sealing the bore 416 by means of the chuck-type mechanism in order to grip the cannula 350 and the tip element 410 may be positioned in an open operative orientation by adjusting the diameter of the bore 416 in order to allow passage of the cannula 350 therethrough.

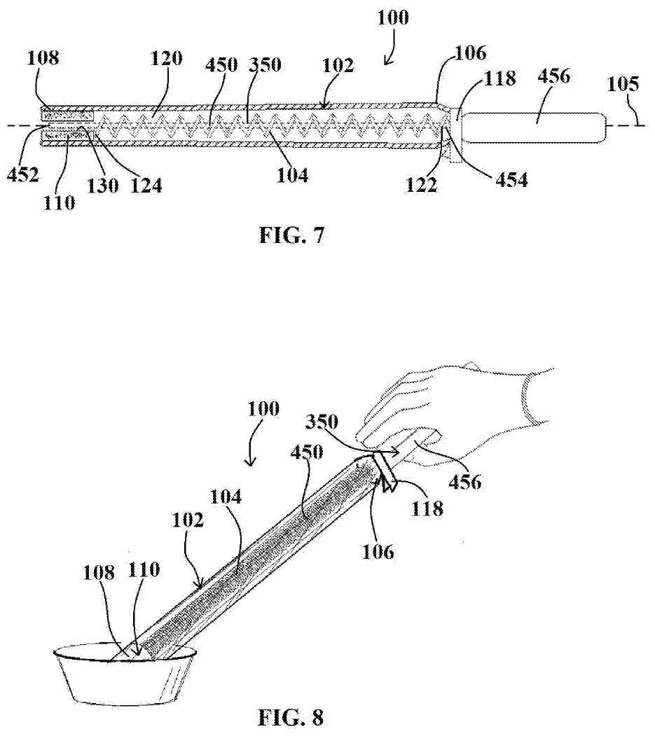

Reference is now made to FIG. 7, which is a simplified sectional illustration of the protective sheathing and lubricating device 100 of FIG. 1, section being taken along lines A-A in FIG. 1.

It is appreciated that a surgical instrument, such as the medical cannula 350, which is depicted in dashed lines in FIG. 7, can be inserted into the protective sheathing and lubricating device 100, which enables performing the entire surgical procedure without compromising the sterile environment of the medical cannula 350, as is described in detail hereinbelow.

It is seen in FIG. 7 that the medical cannula 350 has an elongated shaft 450 having a distal end 452 and a proximal end 454 and a handle 456 fixedly attached to the proximal end 454 of the medical cannula 350.

It is noted that any elongate surgical instrument may be used with the protective sheathing and lubricating device 100 according to an embodiment of the present invention, such as a liposuction cannula, lipoinjection cannula, an endoscope, a drill or a reamer.

It is a particular feature of an embodiment of the present invention that the protective sheathing and lubricating device 100 includes an external collapsible sheath 102 and a tip element 110, which is preferably fixedly attached to the collapsible sheath 102, and a biasing element 104 residing within the internal volume 120 of the collapsible sheath 102. The biasing element 104 in its at-rest operative state is generally of the same length as the collapsible sheath 102, such that both the elongated shaft 450 and the biasing element 104 are covered by the collapsible shaft 102 to their entire longitudinal extent.

It is noted that the tip element 110 is fixedly attached to the collapsible sheath 102 by an adhesive, as mentioned hereinabove. Alternatively, the tip element 110 may be attached to the collapsible sheath 102 by an adhesive tape, welding, or a friction-fit element. Further alternatively, the tip element 110 is fixedly attached to the biasing element 104. It is appreciated that if the tip element 110 is made of a plastic material, it may be fixedly attached to the collapsible sheath 102 by fixation element, such as a grommet, pin or staple. The tip element 110 is preferably used to stabilize the elongated shaft 450 of the cannula element 350 within the protective sheathing and lubricating device 100 and may also be used to disinfect the elongated shaft 450, as is described in detail hereinbelow.

It is further noted that the biasing element 104 is fixedly attached to the tip element 110 by adhesive. Alternatively, the biasing element 104 may be fixedly attached to the inner surface of the collapsible sheath 102 or not be attached at all and remain supported at one of its sides against the tip element at its other side against the handle 456 of the medical cannula 350.

It is a further particular feature of an embodiment of the present invention that the elongated shaft 450 of the medical cannula 350 is inserted into the biasing element 104 of the protective sheathing and lubricating device 100, such that the elongated shaft 450, the biasing element 104 and the collapsible sheath 102 all extend mutually coaxially along longitudinal axis 105. The distal end 452 of the medical cannula 350 preferably extends through the bore 130 of the tip element 110, such that the distal end 108 of the collapsible sheath 102, the distal end of the tip element 110 and the distal end 452 of the elongated shaft 450 are generally aligned. Alternatively, the distal end 452 of the elongated shaft 450 may extend farther distally of the distal end 108 of the collapsible sheath 102 or it may not reach the distal end 108 of the collapsible sheath 102.

It is noted that the distal end of the elongated shaft 450 of the medical cannula 350 is preferably fitted within bore 130 of tip element 110. Alternatively, tip element 110 may be formed without an interior bore and elongated shaft 450 may then penetrate the tip element 110 during distal displacement thereof.

It is a still further particular feature of an embodiment of the present invention that the biasing element 104 may extend through bore 130 of the tip element 110 and may be fixedly attached to the inner surface of the tip element 110, thus preventing dislodging of tip element particles during axial displacement of the medical cannula 350 relative to the tip element 110 during the surgical procedure.

It is appreciated that the proximal end 106 of the collapsible sheath 102 is preferably removably attached to the proximal end 454 of the elongated shaft 450 of the medical cannula 350, such as by means of adhesive tape 118, in order to prevent relative longitudinal displacement between the proximal end 106 of the collapsible sheath 102 and the proximal end 454 of the elongated shaft 450.

It is appreciated that the adhesive tape 118 may be a separate element applied to either the proximal end 106 of the collapsible sheath 102 or to the proximal end 454 of the elongated shaft 450. Alternatively, the adhesive tape 118 may be formed as integral part of the collapsible sheath 102. Further alternatively, the biasing element 104 can be designed to grasp the elongated shaft 450 directly, such as by a tapered expandable proximal end. It is noted that any other type of attachment may be suitable to attach the proximal end 106 of the collapsible sheath 102 and the proximal end 454 of the elongated shaft 450, such as elastic band, clip, clamp or friction-fit element.

It is a particular feature of another embodiment of the present invention that an additional tip element, identical or similar to tip element 110 may be positioned within the interior volume 120 of the collapsible sheath 102 and fixedly attached to the proximal end 106 thereof.

It is appreciated that according to an embodiment of the present invention the collapsible sheath 102 includes demarcation on the outer surface thereof, enabling the user to identify the appropriate length, suitable to the length of the medical cannula 350 to be used during the surgical procedure, and thus enabling the user to cut the collapsible sheath 102 and the biasing element 104 at a location suitable with a particular medical cannula 350.

It is a particular feature of yet another embodiment of the present invention that the protective sheath device 100 includes only the collapsible sheath 102 and the biasing element 104 residing therewithin. The biasing element 104 may be made of a sponge material, which extends from the proximal end 106 of the of the collapsible sheath 102 to the distal end 108 thereof.

It is a particular feature of another embodiment of the present invention that the protective sheath device 100 includes only the biasing element 104 and the tip element 110/210/310/410 attached thereto, without employing a collapsible sheath, such as sheath 102. In a further particular feature of yet another embodiment of the present invention, the protective sheath device 100 includes only the biasing element 104, without employing either a tip element, such as tip element 110/210/310/410 or a collapsible sheath, such as sheath 102.

Reference is now made to FIG. 8, which is a simplified pictorial illustration showing the protective sheathing and lubricating device 100 of FIG. 1, along with a surgical instrument, such as medical cannula 350, inserted thereinto, in a preparatory operative orientation as used by a surgeon.

It is seen in FIG. 8 that the surgeon saturates the tip element 110 of the protective sheathing and lubricating device 100 in a disinfectant, such as iodine, chlorhexidine, stanhexidine, isopropyl or alcohol, before initiation of the surgical procedure, such that once the elongated shaft 450 of the medical cannula 350 extends through the bore 130 of the tip element 110, the elongated shaft 450 passes through the saturated tip element 110 and is thereby disinfected.

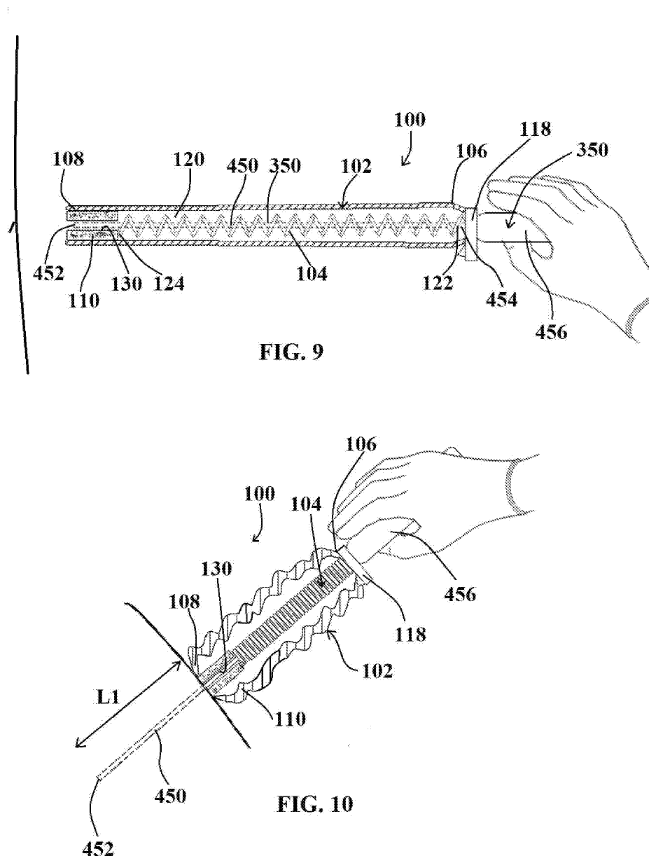

Reference is now made to FIG. 9, which is a simplified sectional illustration showing the protective sheathing and lubricating device 100 of FIG. 1, along with a surgical instrument, such as the medical cannula 350, inserted thereinto, in a pre-insertion operative orientation, section being taken along lines A-A in FIG. 1.

It is seen in FIG. 9 that the surgeon holds the protective sheathing and lubricating device 100 in the vicinity of the surgical site.

It is appreciated that in this pre-insertion operative orientation both the collapsible sheath 102 and the biasing element 104 are positioned in their at-rest operative state, where the entire longitudinal extent of the elongated shaft 450 of the medical cannula 350 is enclosed within the protective sheathing and lubricating device 100 and thus is disposed within a sterile environment and prevents contamination of the cannula.

Reference is now made to FIG. 10, which is a simplified sectional illustration showing the protective sheathing and lubricating device 100 of FIG. 1, along with a surgical instrument, such as the medical cannula 350, inserted thereinto, in a partially-inserted operative orientation as used by the surgeon.

It is seen in FIG. 10 that the surgeon supports the protective sheathing and lubricating device 100 against the surgical site and advances the medical cannula 350 distally through an incision created in the surgical site, such that the elongated shaft 450 of the medical cannula 350 is inserted into the body of the patient to a longitudinal extent depicted as L1 in FIG. 10.

It is appreciated that in this partially-inserted operative orientation the medical cannula 350 is displaced distally against the force of the biasing element 104, the elongated shaft 450 thus extends through the tip element 110/310/410 or penetrates the tip element 210, and both the collapsible sheath 102 and the biasing element 104 are axially compressed and are being positioned in their partially-compressed state as seen in FIG. 10, where the compression extent substantially equals the longitudinal extent L1. It is seen in FIG. 10 that the distal portion of the elongated shaft 450 is disposed within the body of the patient and the remaining proximal portion of the elongated shaft 450 is protected by the partially-compressed protective sheathing and lubricating device 100, thus maintaining the sterile environment of the medical cannula 350.

It is noted that the distal portion of the elongated shaft 450 is disinfected during displacement through the tip element 110/210/310/410 due to contact therewith or due to exudation of the disinfectant from the tip element 110 onto the portion of the elongated shaft 450 passing through the bore 130 as the tip element 110 compresses and is squeezed against the surgical site. The tip element 110/210/310/410 that has been saturated with antiseptic solution is adapted for repeatedly applying antiseptic to the elongated shaft 450 of the medical cannula 350 each time that the elongated shaft 450 is displaced with respect to the tip element 110/210/310/410.

The tip element 110/210/310/410 that has been saturated with antiseptic solution is also adapted to apply the antiseptic solution to the area of skin around the incision site and therefore provides for continuous disinfection of the patient's skin at the surgical site, at and at the vicinity of the incision area, thus reducing infection rate.

It is also a particular feature of an embodiment of the present invention that lubrication of the surgical site and particularly the incision area by means of lubrication of the tip element 110/210/310/410 is configured to decrease or eliminate the problem of friction burn, which is commonly seen on patient's skin during surgical procedures, such as liposuction and fat grafting. During liposuction, the cannula 350 is vigorously displaced through the incision site multiple times and in rapid succession. This leads to friction burns induced by the cannula 350 to the skin around the incision site due to heat generated as well as due to mechanical shearing of the upper layers of skin by the rigid cannula 350. Such friction burns lead to substantial damage to the patient's skin around the incision site which subsequently leads to poor scarring or other healing complications.

The tip element 110/210/310/410, which is soaked in fluid, and particularly a lubricant such as a combined antiseptic/lubricant ointment like iodine-infused gel, preferably provides continuous lubrication and fluid presence at the incision site and at the cannula via direct contact or exudation from the tip element 110/210/310/410, thereby decreasing local heat production and shearing. Lubricated tip element 110/210/310/410 which engages the skin of the patient at the surgical site is thus configured to minimize friction and shearing-induced skin injury and burns at the incision site.

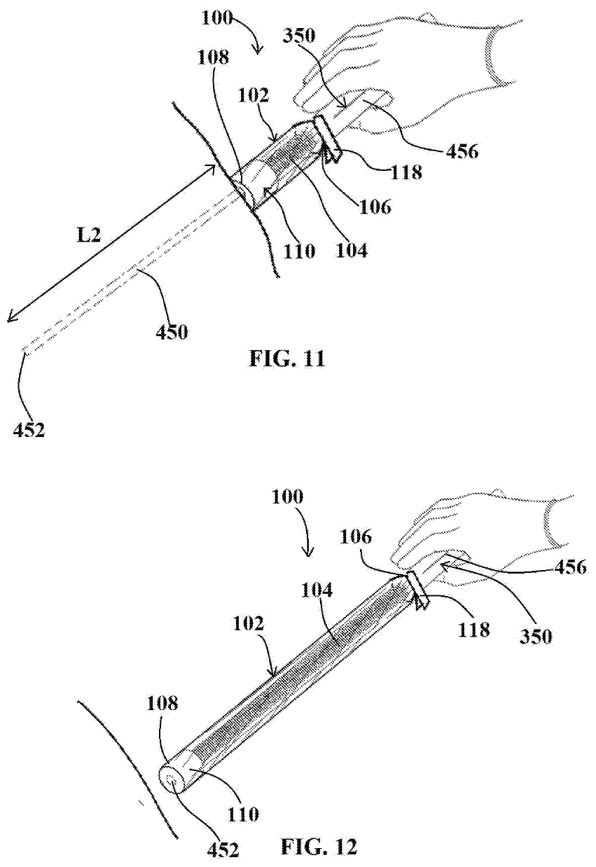

Reference is now made to FIG. 11, which is a simplified pictorial illustration showing the protective sheathing and lubricating device 100 of FIG. 1, along with a surgical instrument, such as the medical cannula 350, inserted thereinto, in a fully-inserted operative orientation as used by the surgeon.

It is seen in FIG. 11 that the surgeon still supports the protective sheathing and lubricating device 100 against the surgical site and further advances the medical cannula 350 distally through the incision created in the surgical site, such that the elongated shaft 450 of the medical cannula 350 is inserted into the body of the patient to a longitudinal extent depicted as L2 in FIG. 11, which is substantially greater than Ll.

It is appreciated that in this fully-inserted operative orientation the medical cannula 350 is further displaced distally against the force of the biasing element 104 and both the collapsible sheath 102 and the biasing element 104 are further axially compressed and are being positioned in their fully-compressed state as seen in FIG. 11, where the compression extent substantially equals the longitudinal extent L2. It is seen in FIG. 11 that the distal portion of the elongated shaft 450 is disposed within the body of the patient and the remaining proximal portion of the elongated shaft 450 is protected by the fully-compressed protective sheathing and lubricating device 100, thus still maintaining the sterile environment of the medical cannula 350.

It is noted that during advancement of the medical cannula 350 through the incision, the tip element 110/210/310/410, which is soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated tip element 110/210/310/410 which engages the skin of the patient at the surgical site is thus configured to minimize friction and shearing-induced skin injury and burns at the incision site.

Reference is now made to FIG. 12, which is a simplified pictorial illustration showing the protective sheathing and lubricating device 100 of FIG. 1, along with a surgical instrument, such as the medical cannula 350, inserted thereinto, in a retracted operative orientation as used by the surgeon.

It is seen in FIG. 12 that the surgeon retracts the protective sheathing and lubricating device 100 from the surgical site by displacing the medical cannula 350 proximally, such that the entire longitudinal extent of the elongated shaft 450 of the medical cannula 350 is once again protected by the protective sheathing and lubricating device 100.

It is appreciated that in this retracted operative orientation the medical cannula 350 is displaced proximally out of the surgical site and once the protective sheathing and lubricating device 100 is no more supported against the surgical site, the collapsible sheath 102 is urged to assume its at-rest operative state under the biasing force of the biasing element 104. It is seen in FIG. 12 that the entire longitudinal extent of the elongated shaft 450 is disposed within the protective sheathing and lubricating device 100, thus maintaining the sterile environment of the medical cannula 350.

It is a particular feature of an embodiment of the present invention that during the retraction of the medical cannula 350 proximally, the distal portion of the elongated shaft 450 of the medical cannula 350 is displaced axially through the saturated tip element 110 and is disinfected after removal from the surgical site due to contact therewith or due to exudation of the disinfectant from the tip element 110 onto the portion of the elongated shaft 450 passing through the bore 130 of the tip element 110.

It is a further particular feature of an embodiment of the present invention that the elongated shaft 450 of the medical cannula 350 is maintained within a sterile environment at all times during the surgical procedure by enclosing the exposed part of the elongated shaft 450 within the protective sheathing and lubricating device 100. This sterile environment is enhanced by means of saturating the tip element 110 in disinfectant before each re-insertion of the elongated shaft 450 into the surgical site and swabbing of the distal portion of the elongated shaft 450 by means of displacement thereof through the saturated tip element 110.

It is also noted that during retraction of the medical cannula 350 through the incision, the tip element 110/210/310/410, which is soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated tip element 110/210/310/410 which engages the skin of the patient at the surgical site is thus configured to minimize friction and shearing-induced skin injury and burns at the incision site.

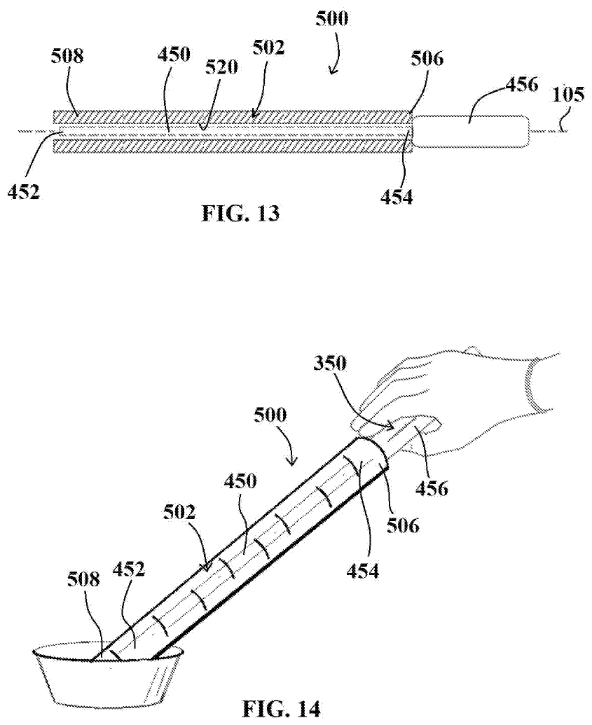

Reference is now made to FIG. 13, which is a simplified sectional illustration of a protective sheathing and lubricating device constructed and operative in accordance with another embodiment of the present invention.

It is a particular feature of another embodiment of the present invention as illustrated specifically in FIG. 13 that a protective sheathing and lubricating device 500 includes a single multi-functional collapsible sheath 502. Specifically, the collapsible sheath 502 has at least one of the following functions: (1) the collapsible sheath 502 is used for covering the elongated shaft 450 which is inserted therethrough; (2) the collapsible sheath 502 is used as a biasing element due to inherent resilient characteristics of the material the collapsible sheath 102 is made of, such as a sponge foam; (3) the collapsible sheath 502 is used as a disinfecting element due to saturation thereof within a disinfecting agent.

It is appreciated that the collapsible sheath 502 may be coated with a waterproof layer of material.

It is seen in FIG. 13 that the collapsible sheath 502 is preferably an integrally formed tubular element defining a proximal end 506, a distal end 508 and an internal volume 520. The collapsible sheath 502 is preferably made of a relatively resilient material, such as rubber or foam.

It is appreciated that a surgical instrument, such as the medical cannula 350, which is depicted in dashed lines in FIG. 13, can be inserted into the protective sheathing and lubricating device 500, which enables performing the entire surgical procedure without compromising the sterile environment of the medical cannula 350, as is described in detail hereinbelow.

It is a particular feature of an embodiment of the present invention that the entire longitudinal extent of the elongated shaft 450 of the medical cannula 350 is covered by the collapsible sheath 502.

It is noted that the proximal end 506 of the collapsible sheath 502 may be attached to the proximal end 454 of the elongated shaft 450 of the medical cannula 350. Alternatively, the collapsible sheath 502 may not be attached to the elongated shaft 450 and remain supported against the handle 456 of the medical cannula 350 during use.

It is a further particular feature of an embodiment of the present invention that the elongated shaft 450 of the medical cannula 350 is inserted into the collapsible sheath 502, such that the elongated shaft 450 and the collapsible sheath 502 extend mutually coaxially along longitudinal axis 105. The elongated shaft 450 extends through the interior volume 520 of the collapsible sheath 502 such that the distal end 508 of the collapsible sheath 502 and the distal end 452 of the elongated shaft 450 are generally aligned. Alternatively, the distal end 452 of the elongated shaft 450 may extend farther distally of the distal end 508 of the collapsible sheath 502 or it may not reach the distal end 508 of the collapsible sheath 502.

It is appreciated that according to an embodiment of the present invention the collapsible sheath 502 includes demarcation on the outer surface thereof, enabling the user to identify the appropriate length, suitable to the length of the medical cannula 350 to be used during the surgical procedure, and thus enabling the user to cut the collapsible sheath 502 at a location suitable with a particular medical cannula 350.

It is noted that at least a portion of the collapsible sheath 502 is adapted to be saturated with antiseptic solution, thus providing for disinfection of the elongated shaft 450 during displacement thereof within the collapsible sheath 502 due to contact therewith or due to exudation of the disinfectant from the collapsible sheath 502 onto the portion of the elongated shaft 450 passing through the collapsible sheath 502 as it compresses and is squeezed against the surgical site.

At least a portion of the collapsible sheath 502 that has been saturated with antiseptic solution is adapted for repeatedly applying antiseptic to the elongated shaft 450 of the medical cannula 350 each time that the elongated shaft 450 is displaced with respect to the collapsible sheath 502.

The collapsible sheath 502 that has been saturated with antiseptic solution is also adapted to apply the antiseptic solution to the area of skin around the incision site and therefore provides for continuous disinfection of the patient's skin at the surgical site, at and at the vicinity of the incision area, thus reducing infection rate.

It is also a particular feature of an embodiment of the present invention that lubrication of the surgical site and particularly the incision area by means of lubrication of at least a portion of the collapsible sheath 502 is configured to decrease or eliminate the problem of friction burn, which is commonly seen on patient's skin during surgical procedures, such as liposuction and fat grafting. During liposuction, the cannula 350 is vigorously displaced through the incision site multiple times and in rapid succession. This leads to friction burns induced by the cannula 350 to the skin around the incision site due to heat generated as well as due to mechanical shearing of the upper layers of skin by the rigid cannula 350. Such friction burns lead to substantial damage to the patient's skin around the incision site which subsequently leads to poor scarring or other healing complications.

The collapsible sheath 502, which is at least partially soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated collapsible sheath 502 which engages the skin of the patient at the surgical site is thus configured to minimize friction and shearing-induced skin injury and burns at the incision site.

Reference is now made to FIG. 14, which is a simplified pictorial illustration showing the protective sheathing and lubricating device 500 of FIG. 13, along with a surgical instrument, such as medical cannula 350, inserted thereinto, in a preparatory operative orientation as used by a surgeon.

It is seen in FIG. 14 that the surgeon saturates the distal end 508 of the collapsible sheath 502 in a disinfectant, such as iodine, before initiation of the surgical procedure, such that once the elongated shaft 450 of the medical cannula 350 is displaced through the collapsible sheath 502, it is disinfected.

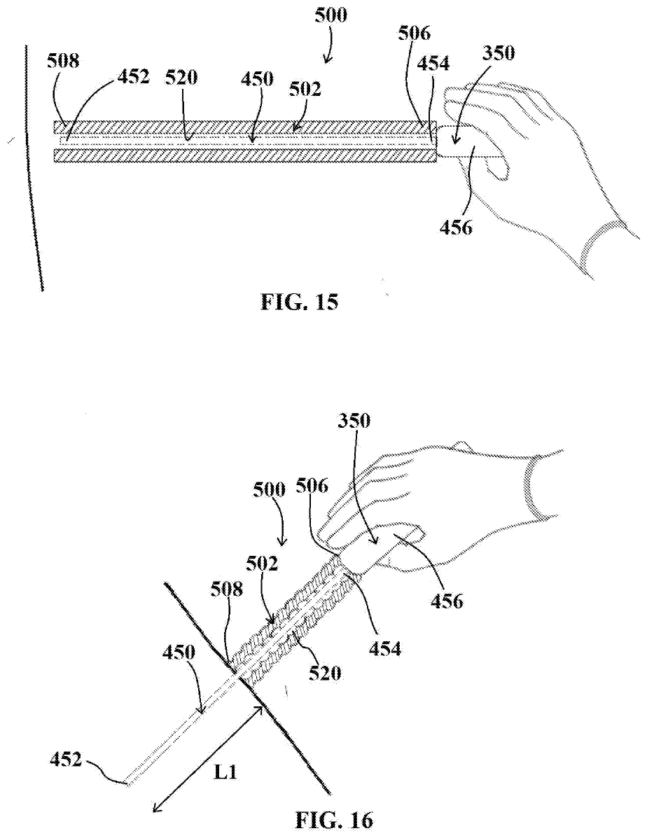

Reference is now made to FIG. 15, which is a simplified sectional illustration showing the protective sheathing and lubricating device 500 of FIG. 13, along with a surgical instrument, such as the medical cannula 350, inserted thereinto, in a pre-insertion operative orientation, as used by the surgeon.

It is seen in FIG. 15 that the surgeon holds the protective sheathing and lubricating device 500 in the vicinity of the surgical site.

It is appreciated that in this pre-insertion operative orientation the collapsible sheath 502 is positioned in its at-rest operative state, where the entire longitudinal extent of the elongated shaft 450 of the medical cannula 350 is enclosed within the protective sheathing and lubricating device 500 and thus is disposed within a sterile environment.

Reference is now made to FIG. 16, which is a simplified sectional illustration showing the protective sheathing and lubricating device 500 of FIG. 13, along with a surgical instrument, such as the medical cannula 350, inserted thereinto, in a partially-inserted operative orientation as used by the surgeon.

It is seen in FIG. 16 that the surgeon supports the protective sheathing and lubricating device 500 against the surgical site and advances the medical cannula 350 distally through an incision created in the surgical site, such that the elongated shaft 450 of the medical cannula 350 is inserted into the body of the patient to a longitudinal extent depicted as L1 in FIG. 16.

It is appreciated that in this partially-inserted operative orientation the medical cannula 350 is displaced distally against the force of the collapsible sheath 502, which serves as the biasing element. The elongated shaft 450 thus extends through the collapsible sheath 502, which is axially compressed and positioned in its partially-compressed state, where the compression extent substantially equals the longitudinal extent L1. It is seen in FIG. 16 that the distal portion of the elongated shaft 450 is disposed within the body of the patient and the remaining proximal portion of the elongated shaft 450 is protected by the partially-compressed protective sheathing and lubricating device 500, thus maintaining the sterile environment of the medical cannula 350.

It is noted that the distal portion of the elongated shaft 450 is disinfected during displacement through the saturated collapsible sheath 502 due to contact therewith or due to exudation of the disinfectant from the collapsible sheath 502 onto the portion of the elongated shaft 450 passing through the collapsible sheath 502 as it compresses and is squeezed against the surgical site.

It is noted that during advancement of the medical cannula 350 through the incision, the collapsible sheath 502, which is at least partially soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated distal end 508 of the collapsible sheath 502 which engages the skin of the patient at the surgical site is thus configured to minimize friction and shearing-induced skin injury and burns at the incision site.

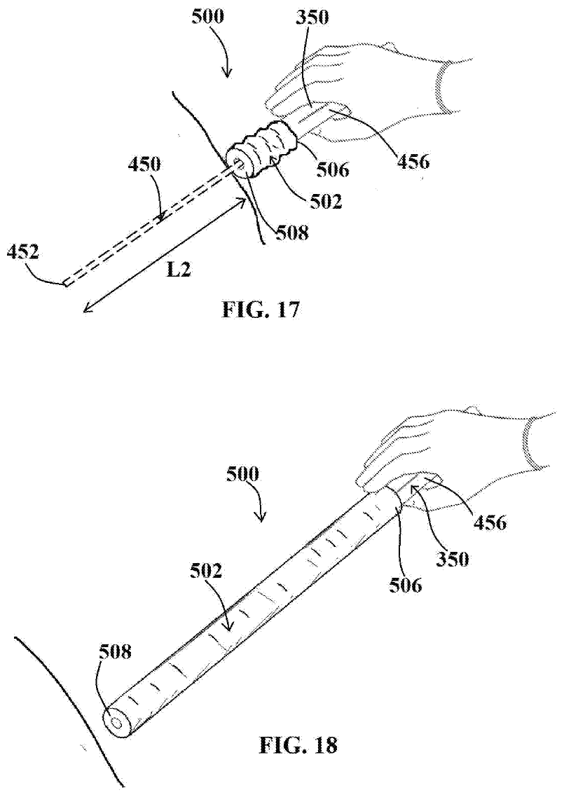

Reference is now made to FIG. 17, which is a simplified pictorial illustration showing the protective sheathing and lubricating device 500 of FIG. 13, along with a surgical instrument, such as the medical cannula 350, inserted thereinto, in a fully-inserted operative orientation as used by the surgeon.

It is seen in FIG. 17 that the surgeon still supports the protective sheathing and lubricating device 500 against the surgical site and further advances the medical cannula 350 distally through the incision created in the surgical site, such that the elongated shaft 450 of the medical cannula 350 is inserted into the body of the patient to a longitudinal extent depicted as L2 in FIG. 17, which is substantially greater than Ll.

It is appreciated that in this fully-inserted operative orientation the medical cannula 350 is further displaced distally against the force of the collapsible sheath 502, which is further axially compressed and is being positioned in its fully-compressed state as seen in FIG. 17, where the compression extent substantially equals the longitudinal extent L2. It is seen in FIG. 17 that the distal portion of the elongated shaft 450 is disposed within the body of the patient and the remaining proximal portion of the elongated shaft 450 is protected by the fully-compressed protective sheathing and lubricating device 500, thus still maintaining the sterile environment of the medical cannula 350.

It is noted that during further advancement of the medical cannula 350 through the incision, the collapsible sheath 502, which is at least partially soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated distal end 508 of the collapsible sheath 502 which engages the skin of the patient at the surgical site is thus configured to minimize friction and shearing-induced skin injury and burns at the incision site.

Reference is now made to FIG. 18, which is a simplified pictorial illustration showing the protective sheathing and lubricating device 500 of FIG. 13, along with a surgical instrument, such as the medical cannula 350, inserted thereinto, in a retracted operative orientation as used by the surgeon.

It is seen in FIG. 18 that the surgeon retracts the protective sheathing and lubricating device 500 from the surgical site by displacing the medical cannula 350 proximally, such that the entire longitudinal extent of the elongated shaft 450 of the medical cannula 350 is once again protected by the protective sheathing and lubricating device 500.

It is appreciated that in this retracted operative orientation the medical cannula 350 is displaced proximally out of the surgical site and once the protective sheathing and lubricating device 500 is no more supported against the surgical site, the collapsible sheath 502 is urged to assume its at-rest operative state due to its inherent resilient characteristics. It is seen in FIG. 18 that the entire longitudinal extent of the elongated shaft 450 is disposed within the protective sheathing and lubricating device 500, thus maintaining the sterile environment of the medical cannula 350.

It is a particular feature of an embodiment of the present invention that during the retraction of the medical cannula 350 proximally, the distal portion of the elongated shaft 450 of the medical cannula 350 is displaced axially through the saturated collapsible sheath 502 and is disinfected after removal from the surgical site due to contact therewith or due to exudation of the disinfectant from the collapsible sheath 502 onto the portion of the elongated shaft 450 passing through the interior volume 520 of the collapsible sheath 502.

It is a further particular feature of an embodiment of the present invention that the elongated shaft 450 of the medical cannula 350 is maintained within a sterile environment at all times during the surgical procedure by enclosing the exposed part of the elongated shaft 450 within the protective sheathing and lubricating device 500. This sterile environment is enhanced by means of saturating the collapsible sheath 502 in disinfectant before each re-insertion of the elongated shaft 450 into the surgical site and swabbing of the distal portion of the elongated shaft 450 by means of displacement thereof through the saturated collapsible sheath 502.

It is noted that during retraction of the medical cannula 350 through the incision, the collapsible sheath 502, which is at least partially soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated distal end 508 of the collapsible sheath 502 which engages the skin of the patient at the surgical site is thus configured to minimize friction and shearing-induced skin injury and burns at the incision site.

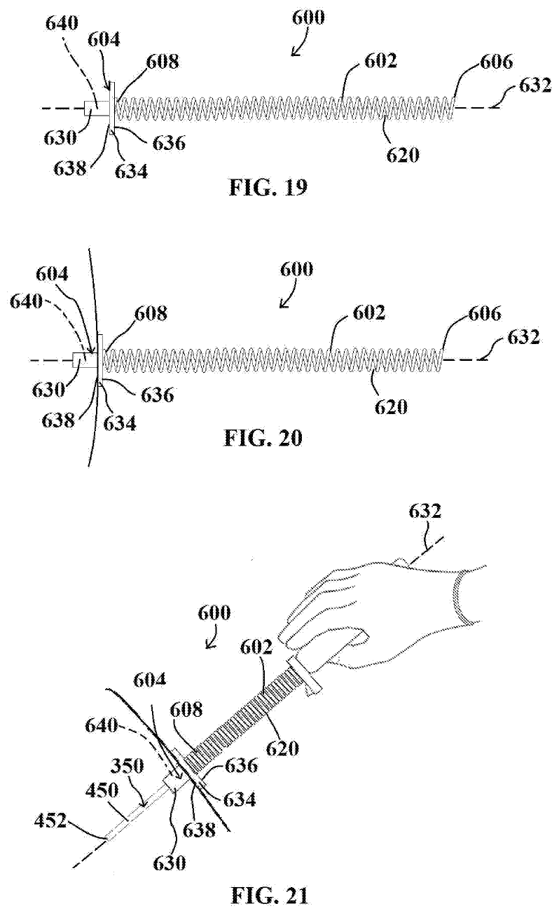

Reference is now made to FIG. 19, which is a simplified pictorial illustration of a protective sheathing and lubricating device constructed and operative in accordance with still another embodiment of the present invention.

It is a particular feature of still another embodiment of the present invention as illustrated specifically in FIG. 19 that a protective sheathing and lubricating device 600 includes a biasing element 602, which is attached to or integrally made with a tip element 604.

It is appreciated that the sheathing and lubricating device 600 may alternatively or additionally include any of the components described with respect to FIGS. 1-18. For example, sheathing and lubricating device 600 may additionally include a collapsible sheath mounted over the biasing element 602.

The sheathing and lubricating device 600 has at least one of the following functions: (1) covering the elongated shaft 450 of the medical cannula 350 (not shown), which is inserted therethrough; (2) biasing element, due to inherent resilient characteristics of the material the biasing element 602 is made of, such as a sponge foam; (3) the tip element 604 is used as a disinfecting element due to saturation thereof within a disinfecting agent; (4) preventing friction burns at the incision site by providing a barrier between the cannula 350 (not shown) and the patient's skin during the surgical procedure, by means of at least partially inserting the tip element 604 into the incision site.

It is noted that skin ports are currently used for liposuction procedures, whereas the skin ports are inserted into the incision site and sutured onto the surrounding skin to protect the skin from damage caused by repetitive movement of the medical cannula through the incision site. This type of skin port requires a separate port at every incision site and thus plurality of such skin ports usually have to be used during a single surgical procedure.

It is a particular feature of an embodiment of the present invention that the tip element 604 is used as an alternative to tip elements 110/210/310/410, as described hereinabove and provides for both: (1) disinfecting the elongated shaft 450 of the medical cannula 350 due to saturation thereof in an antiseptic solution; and (2) preventing friction burns by means of partial insertion of the tip element 604 into the incision site and thus providing a mechanical barrier between the patient's skin and the elongated shaft 450 of the medical cannula 350.

It is a further particular feature of an embodiment of the present invention that the tip element 604, being an integral part of the sheathing and lubricating device 600, which is mounted over the medical cannula 350, provides for continuous application of lubricating fluids, gels or other materials to the skin in and around an incision site and the medical cannula 350 itself by saturating the tip element 604 and/or coating the biasing element 602 and/or the inner surface of the outer collapsible sheath, such as 102 for example, in these materials.

It is appreciated that mounting of a single sheathing and lubricating device 600 onto the medical cannula 350 provides for friction burn prevention at each incision site that the cannula 350 traverses. Furthermore, the lubricant material can be applied in sufficient quantities such that the tip element 604 and the biasing element 602 act as a reservoir for the lubricant material, thereby diminishing or eliminating the need to reapply lubricant throughout the surgical procedure.

It is a particular feature of an embodiment of the present invention that the sheathing and lubricating device 600 preferably includes any of the following component variations or combinations thereof: (1) biasing element 602/502/104 only without employing a tip element, whereas the biasing element is preferably coated in anti-septic fluid or gel; (2) biasing element 602/502/104 with a collapsible sheath, such as sheath 102, mounted over the biasing element, but without employing a tip element; (3) biasing element 602/502/104 and a tip element, such as 604/110/210/310/410 attached thereto or integrally made therewith; or (4) biasing element 602/502/104, with collapsible sheath 102 mounted thereover and tip element 604/110/210/310/410 attached thereto or integrally made therewith.

It is seen in FIG. 19 that the biasing element 602 is preferably an integrally formed element, such as a compression coil spring, defining a proximal end 606, a distal end 608 and an internal volume 620 for passage of the medical cannula 350 therethrough.

It is a particular feature of an embodiment of the present invention that the entire longitudinal extent of the elongated shaft 450 of the medical cannula 350 is covered by the sheathing and lubricating device 600.

It is noted that at least a portion of the tip element 604, optionally also a portion of the biasing element 602, is adapted to be saturated in antiseptic solution, thus providing for disinfection of the elongated shaft 450 during displacement thereof within the sheathing and lubricating device 600 due to contact therewith or due to exudation of the disinfectant from the sheathing and lubricating device 600 onto the portion of the elongated shaft 450 passing through the sheathing and lubricating device 600 as it compresses and is squeezed against the surgical site.

At least a portion of the tip element 604 that has been saturated with antiseptic solution is adapted for repeatedly applying antiseptic to the elongated shaft 450 of the medical cannula 350 each time that the elongated shaft 450 is displaced with respect to the tip element 604.

The tip element 604 that has been saturated with antiseptic solution is also adapted to apply the antiseptic solution to the area of skin around the incision site and therefore provides for continuous disinfection of the patient's skin at the surgical site, at and at the vicinity of the incision area, thus reducing infection rate.

It is also a particular feature of an embodiment of the present invention that lubrication of the surgical site and particularly the incision area by means of lubrication of at least a portion of the tip element 604 is configured to decrease or eliminate the problem of friction burns, as described in detail hereinabove.

The tip element 604, which is at least partially soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated tip element 604 which engages the skin of the patient at the surgical site is thus configured to minimize friction and shearing-induced skin injury and burns at the incision site.

It is particularly seen in FIG. 19 that tip element 604 includes a generally cylindrical portion 630 preferably extending coaxially with the biasing element 602 along longitudinal axis 632, the cylindrical portion 630 is preferably adapted for insertion into the incision site. Tip element 604 also includes an annular portion 634 extending radially outwardly from the cylindrical portion 630 and arranged transversely with respect to longitudinal axis 632. Annular portion 634 has a proximally facing surface 636 and a distally facing surface 638, which is preferably adapted for engagement with the skin of the patient.

Reference is now made to FIG. 20, which is a simplified pictorial illustration showing the protective sheathing and lubricating device 600 of FIG. 19, in a preparatory operative orientation as used by a surgeon.

It is appreciated that the surgeon saturates the tip element 604 of the sheathing and lubricating device 600 in a disinfectant, such as iodine, before initiation of the surgical procedure, such that once the elongated shaft 450 of the medical cannula 350 is displaced through the sheathing and lubricating device, it is disinfected.

It is seen in FIG. 20 that the surgeon inserts the cylindrical portion 630 of the tip element 604 into the incision site and supports the annular portion 634 of the tip element 604 against the surgical site, such that the distally facing surface 638 engages the skin of the patient.

Reference is now made to FIG. 21, which is a simplified sectional illustration showing the protective sheathing and lubricating device 600 of FIG. 19, along with a surgical instrument 350 inserted thereinto, in a partially-inserted operative orientation as used by a surgeon.

It is seen in FIG. 21 that the medical cannula 350 is advanced distally through an incision created in the surgical site, such that the elongated shaft 450 of the medical cannula 350 is partially inserted into the body of the patient.

It is appreciated that in this partially-inserted operative orientation the medical cannula 350 is displaced distally against the force of the biasing element 602. The elongated shaft 450 thus extends through the biasing element 602 and the tip element 604, the biasing element 602 is axially compressed and positioned in its partially-compressed state. It is seen in FIG. 21 that the distal portion of the elongated shaft 450 is disposed within the body of the patient and the remaining proximal portion of the elongated shaft 450 is protected by the partially-compressed sheathing and lubricating device 600, thus maintaining the sterile environment of the medical cannula 350.

It is noted that the distal portion of the elongated shaft 450 is disinfected during displacement through the saturated tip element 604 due to contact therewith or due to exudation of the disinfectant from the tip element 604 onto the portion of the elongated shaft 450 passing through the tip element 604.

It is noted that during advancement of the medical cannula 350 through the incision, the tip element 604, which is at least partially soaked in fluid, and particularly a lubricant such as a combined antiseptic/lubricant ointment like iodine-infused gel, preferably provides continuous lubrication and fluid presence at the incision site, thereby decreasing local heat production and shearing. Lubricated tip element 604 which engages the skin of the patient at the surgical site is thus configured to minimize friction and shearing-induced skin injury and burns at the incision site. The insertion of the cylindrical portion 630 of the tip element 604 into the incision site also provides for a mechanical barrier between the skin of the patient and the cannula 350, thus even further prevents friction burns on the skin of the patient.

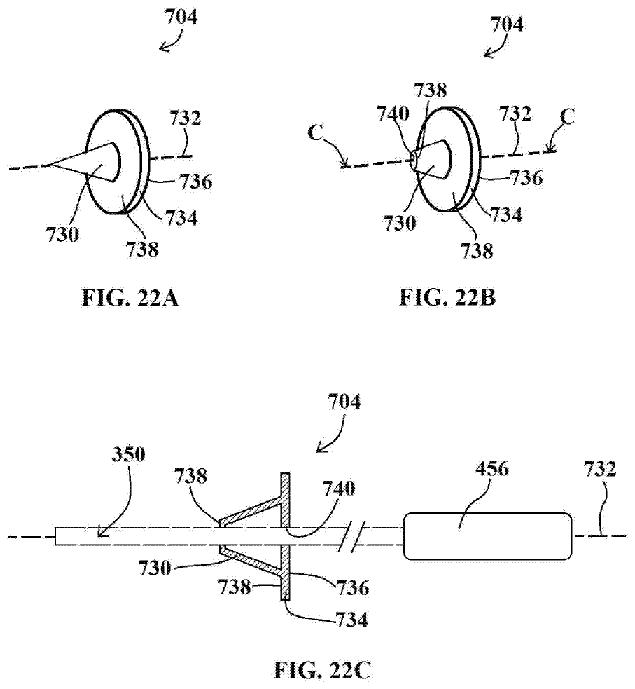

Reference is now made to FIGS. 22A-22C, which are respective two simplified pictorial illustrations and a sectional illustration of a tip element forming part of the protective sheathing and lubricating device 600 of FIG. 19, according to another embodiment of the present invention, the tip element is shown in a preparatory operative orientation, and in a ready for use operative orientation respectively in the pictorial illustration, and the tip element is shown in use in the sectional illustration, section being taken along lines C-C in FIG. 22B.

In accordance with another embodiment of the present invention, an alternative tip element 704 can be used with the sheathing and lubricating device 600, as described with respect to FIGS. 19-21, instead of tip element 604.

It is seen in FIG. 22A that tip element 704 includes a generally conical portion 730, which preferably tapers distally and arranged along a longitudinal axis 732, the conical portion 730 is preferably adapted for insertion into the incision site. Tip element 704 also includes an annular portion 734 extending radially outwardly from the conical portion 730 and arranged transversely with respect to longitudinal axis 732. Annular portion 734 has a proximally facing surface 736 and a distally facing surface 738, which is preferably adapted for engagement with the skin of the patient.

It is seen in FIGS. 22B & 22C that the conical portion 730 of the tip element 704 is cut to the required length for forming an opening that complies with the desired cannula diameter. Following cutting of the conical portion 730, a truncated cone is formed, defining a circular edge 738 at the distal end thereof. A through bore 740 extends through the truncated cone and the annular portion 734, the bore 740 being adapted for unencumbered passage of the medical cannula 350 therethrough.

The tip element 704 is shown prior being cut to length in FIG. 22A and after being cut to length in FIGS. 22B & 22C. Tip element 704 after being cut to the appropriate length is adapted to be inserted into the incision site and be held in place in the incision by the forward pressure exerted by the biasing element 602 on the tip element 704. As the cannula passes into the incision site, the skin in and around the incision site is protected from the forces applied by the cannula displacement by means of the physical barrier between the cannula 350 and the skin provided by the tip element 704.

It is appreciated that tip element 704 is preferably made of rubber, silicone, plastic or other suitable material.

It is further appreciated that the tip element 704 may be a separate element affixed to the biasing element 602 or an integral part of the biasing element 602, such that the biasing element 602 is a resilient spring-like element, made of silicone or nylon, which terminates in a conical tip element 704.

It will be appreciated by persons skilled in the art that the present invention is not limited by what has been particularly shown and described hereinabove.

Rather the scope of the present invention includes both combinations and subcombinations of various features described hereinabove as well as variations and modifications thereof which are not in the prior art.

1. A protective sheathing and lubricating device for use with a surgical instrument, comprising:
a collapsible sheath arranged along a longitudinal axis and having a distal end, a proximal end and an interior volume;
said collapsible sheath is configured to be axially compressible upon exertion of force onto said proximal end;
and wherein said collapsible sheath is further configured to automatically assume an at-rest operative state upon release of said force.

2. A protective sheathing and lubricating device for use with a surgical instrument according to claim 1 and also comprising a biasing element, which extends within the interior volume of said collapsible sheath along said longitudinal axis.

3. A protective sheathing and lubricating device for use with a surgical instrument according to claim 2 and also comprising a tip element, which is fixedly attached to at least one of said distal end and said proximal end of said collapsible sheath.

4. A protective sheathing and lubricating device for use with a surgical instrument according to claim 2 and wherein said biasing element is fixedly attached to said tip element.

5. A protective sheathing and lubricating device for use with a surgical instrument according to claim 1 and wherein said collapsible sheath is made of a resilient material.

6. A protective sheathing and lubricating device for use with a surgical instrument according to claim 2 and wherein said biasing element is a compression spring.

7. A protective sheathing and lubricating device for use with a surgical instrument according to claim 2 and wherein said biasing element is a mesh sleeve.

8. A protective sheathing and lubricating device for use with a surgical instrument according to claim 3 and wherein said tip element is a sponge foam.

9. (canceled)

10. (canceled)

11. (canceled)

12. (canceled)

13. (canceled)

14. (canceled)

15. (canceled)

16. A protective sheathing and lubricating device for use with a surgical instrument, comprising:
a biasing element arranged along a longitudinal axis and having a distal end and a proximal end;
a tip element coupled with said biasing element; and wherein said biasing element is configured to be axially compressible upon exertion of force onto said proximal end.

17. A protective sheathing and lubricating device for use with a surgical instrument according to claim 16 and wherein said tip element has an annular portion and an elongated portion extending generally transversely with respect to said annular portion.

18. A protective sheathing and lubricating device for use with a surgical instrument according to claim 17 and wherein said elongated portion is conical.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A method of disinfecting or lubricating a surgical instrument during insertion thereof into a surgical site, comprising the steps of:
providing a protective sheathing and lubricating device, having an elongate element arranged along a longitudinal axis and having a distal end and a proximal end;
saturating at least a portion of said sheathing and lubricating device within a disinfecting agent;
inserting at least a portion of said surgical instrument into said elongate element, such that at least a portion of said surgical instrument is disinfected by said protective sheathing and lubricating device.

42. A method of disinfecting or lubricating a surgical instrument during insertion thereof into a surgical site according to claim 41 and wherein said protective sheathing and lubricating device also comprises a tip element, which is coupled to said elongate element.

43. A method of disinfecting or lubricating a surgical instrument during insertion thereof into a surgical site according to claim 42 and wherein said disinfection occurs by means of contact between said at least portion of surgical instrument and at least a portion of said tip element.

44. A method of disinfecting or lubricating a surgical instrument during insertion thereof into a surgical site according to claim 43 and wherein said disinfection occurs by means of exudation of said disinfecting agent from at least a portion of said tip element to said at least portion of said surgical instrument.

45. A method of disinfecting or lubricating a surgical instrument during insertion thereof into a surgical site according to claim 42 and wherein said tip element is adapted to be at least partially inserted into said surgical site.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A protective sheathing and lubricating device for use with a surgical instrument according to claim 16 and wherein said biasing element is made of a resilient material.

64. A method of disinfecting or lubricating a surgical instrument during insertion thereof into a surgical site according to claim 41 and wherein said elongate element is made of a resilient material.

65. A method of disinfecting or lubricating a surgical instrument during insertion thereof into a surgical site according to claim 41 and wherein said tip element is a sponge foam.
